(19) United States
(12) Patent Application Publication
(10) Pub. No.: US 2006/0233732 A1
(43) Pub. Date: Oct. 19, 2006

(54) Keratin fiber coating composition comprising a continuous aqueous phase and at least one volatile oil

(76) Inventor: Nathalie Jager Lezer, Verieres-le-Buisson (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/389,126

(22) Filed: Mar. 27, 2006

Related U.S. Application Data
(60) Provisional application No. 60/667,070, filed on Apr. 1, 2005.

(30) Foreign Application Priority Data
Mar. 25, 2005 (FR) 05 50786

(51) Int. Cl. A61K 8/89

(52) U.S. Cl. 424/70.7

(57) ABSTRACT

The present disclosure relates to a keratin fiber coating composition comprising a continuous aqueous phase and at least one volatile oil, wherein when the composition forms a film on the keratin fibers, it has a water resistance such that ΔL is greater than or equal to 4.5 and/or a sebum resistance such that ΔL is greater than or equal to −2.5.

This application claims benefit of U.S. Provisional Application No. 60/667,070, filed Apr. 1, 2005, the contents of which are incorporated herein by reference. This application also claims benefit of priority under 35 U.S.C. § 119 to French Patent Application No. FR 05 50786, filed Mar. 25, 2005, the contents of which are also incorporated herein by reference.

The present disclosure relates to making up keratin fibers, for instance the eyelashes, the eyebrows and the hair, and for example, in one embodiment, making up the eyelashes.

The composition according to the present disclosure may be in the form of a product for the eyelashes, or mascara, a product for the eyebrows or a hair makeup product. For instance, one embodiment of the present disclosure relates to a mascara. For example, it may be a makeup composition, a transparent or colored composition to be applied over or under a makeup, also known, respectively, as a “top coat” or a “base coat,” or alternatively an eyelash treatment composition.

In general, compositions for making up keratin fibers, such as the eyelashes, of “emulsion mascara” type are in the form of an emulsion of waxes in an aqueous phase.

It is known practice to use, with waxes, film-forming polymers, which may be dissolved or dispersed in an aqueous medium, as described in documents FR-A-2 528 699 and EP-A-0 655 234. U.S. Pat. No. 6,497,861 describes cosmetic compositions, such as mascaras, with a volumizing effect, comprising an aqueous phase and an oily phase comprising a volatile oil gelled with a polyamide resin.

However, the makeup film obtained after applying these compositions may not always be sufficiently water-resistant, for example when bathing or taking showers, or to tears, sweat or sebum. The mascara can then have a tendency to become worn away over time: grains can become deposited and unattractive marks can appear around the eyes.

Documents EP 0 388 582 and WO 94/17775 also disclose mascara compositions comprising an aqueous phase, a film-forming polymer and a volatile oil, which are capable of forming on keratin fibers, after evaporating off the volatile oil, a film that has good staying power by virtue of a high amount of film-forming polymer. However, the presence of a high amount of film-forming polymer can have drawbacks reflected by a pasty texture of the composition, which can form, after being deposited on the keratin fibers, a granular, non-uniform film that lacks slipperiness on application.

Thus there is a need in the art for compositions that are long lasting and water- and sebum-resistant, which do not become granular, and which are easy to apply. The inventors have discovered, unexpectedly, that the incorporation of at least one volatile oil into a composition with a continuous aqueous phase makes it possible to improve the properties of the composition, for example in terms of water resistance and sebum resistance. In addition, the compositions according to the present disclosure have a satisfactory viscosity that allows the deposition of a smooth, uniform film on keratin fibers and lead to a charging (or volumizing) effect on the said keratin fibers.

As used herein, the term “composition with a continuous aqueous phase” is understood to mean that the composition has a conductivity, measured at 25° C., of greater than 23 μS/cm (microSiemens/cm), the conductivity being measured, for example, using an MPC227 conductimeter from Mettler Toledo and an Inlab 730 conductivity measuring cell. The measuring cell is immersed in the composition, so as to remove the air bubbles liable to form between the two electrodes of the cell. The conductivity reading is taken once the conductimeter value has stabilized. An average is determined on at least three successive measurements.

Therefore, the present disclosure proposes a novel route for formulating a keratin fiber coating composition that has good properties of water and/or sebum resistance, and which solves all or some of the problems associated with the conventional formulation routes.

For instance, one aspect of the present disclosure is a cosmetic keratin fiber coating composition comprising a continuous aqueous phase and at least one volatile oil, wherein when the composition forms a film, the film has a water resistance such that ΔL is greater than or equal to −4.5, the at least one volatile oil being chosen from isododecane, 3-butyl-1,1,1,3,5,5,5-heptamethyltrisiloxane, 3-ethyl-1,1,1,3,5,5,5-heptamethyltrisiloxane and octamethyltrisiloxane.

The water resistance of the composition, represented by ΔL, is, for example, such that ΔL ranges from −4.5 to 0, for example ΔL is greater than or equal to −4, for example ranging from −4 to −0.1, and such as ΔL is greater than or equal to −2.5, for example ranging from −2.5 to −0.2.

According to the present disclosure, the term “water resistance” is understood to mean the in vitro water resistance evaluated by colorimetry according to the following protocol:

The composition is applied to three samples of 30 knots straight Caucasian hair (60 eyelashes 1 cm long), 2 cm fringe length, by performing three sets of 10 sweeps with two-minute intervals, with uptake of product between each series of 10 sweeps. Each sample is then dried at room temperature for a drying time of one hour.

The three made-up samples are immersed in a container containing water, for one hour. The three samples are moved to and fro five times over a Wypall L40 type square wipe from Kimberley-Clark.

The intensity of black deposited by the sample is then measured using a CR 300 colorimeter from Minolta.

Three measurements are taken on each mascara line and they are then averaged. A coefficient that represents the luminosity (ΔL) is then used.

To avoid variations in color of the support, the measurement is taken as a “reference measurement”: the color of the wipe is used as reference white.

The measurement taken on the clorimeter gives an indicative measurement of the “blackness” of the mascara line: the blacker the line, the further from zero the value (ΔL). In other words, the closer the value (ΔL) is to zero, the better the resistance, and vice versa.

For example, the composition according to the present disclosure is capable of forming a film with a sebum resistance such that ΔL is greater than or equal to −2.5, ranging, for example, from −2.5 to 0, and such as ΔL is greater than or equal to −2.4, for example ranging from −2.4 to −1.5.

According to one embodiment of the present disclosure is a cosmetic keratin fiber coating composition comprising a continuous aqueous phase and at least one volatile oil, wherein when the composition forms a film, the film has a sebum resistance such that ΔL is greater than or equal to −2.5, where the at least one volatile oil is chosen from isododecane, 3-butyl-1,1,1,3,5,5,5-heptamethyltrisiloxane, 3-ethyl-1,1,1,3,5,5,5-heptamethyltrisiloxane and octamethyltrisiloxane.

According to the present disclosure, the term “sebum resistance” is understood to mean the in vitro sebum resistance evaluated by colorimetry according to the same measuring protocol as for the water resistance described above, except that the three made-up samples are immersed in a container containing squalene (squalene is present at 18% in the composition of sebum) instead of water.

Another aspect of the present disclosure is also the use of at least one volatile oil chosen from isododecane, 3-butyl-1,1,1,3,5,5,5-heptamethyltrisiloxane, 3-ethyl-1,1,1,3,5,5,5-heptamethyltrisiloxane and octamethyltrisiloxane, in a keratin fiber coating composition comprising a continuous aqueous phase, to obtain a composition that, when it forms a film deposited on keratin fibers, the film has a water resistance of greater than or equal to −4.5 and/or a sebum resistance such that ΔL is greater than or equal to −2.5.

According to another embodiment, the present disclosure relates to a keratin fiber coating composition comprising a continuous aqueous phase and at least one volatile oil, wherein when the composition forms a film, the film has a water resistance of greater than or equal to −4.5, and the composition having a viscosity, measured at 25° C., of less than or equal to 30 Pa·s.

The viscosity of the composition can range, for example, from 3 to 30 Pa·s, such as from 5 to 15 Pa·s and for example from 7 to 12 Pa·s.

The viscosity of the composition is measured at 25° C. using a Rheomat 180 viscometer (from the company Lamy) equipped with an MS-R1, MS-R2, MS-R3, MS-R4 or MS-R5 spindle chosen as a function of the consistency of the composition, rotating at a spin speed of 200 rpm. The measurement is taken after 10 minutes of rotation. The viscosity measurements are taken not more than one week after manufacture.

Another aspect of the present disclosure is a keratin fiber coating composition comprising a continuous aqueous phase and at least one volatile oil, wherein when the composition forms a film, the film has a sebum resistance such that ΔL is greater than or equal to −2.5, and the composition has a viscosity of less than or equal to 30 Pa·s.

Another embodiment of the present disclosure is also a process for making up keratin fibers, in which a composition as defined above is applied to the keratin fibers, such as to the eyelashes.

According to the present disclosure, the term “volatile organic solvent or oil” is understood to mean an organic solvent or oil (or non-aqueous medium) capable of evaporating on contact with keratin fibers in less than one hour, at room temperature and atmospheric pressure. The volatile oil is a volatile cosmetic oil, which is liquid at room temperature, for example having a non-zero vapor pressure, at room temperature and atmospheric pressure, and further, for example having a vapor pressure ranging from 0.13 Pa to 40,000 Pa (10⁻³ to 300 mmHg) such as ranging from 1.3 Pa to 8,000 Pa (0.01 to 60 mmHg).

As used herein, the expression “at least one” is understood to mean one or more individual compounds, and also mixtures thereof.

The composition according to the present disclosure comprises a physiologically acceptable medium, for example a cosmetically acceptable medium, i.e. a medium that is compatible with keratin fibers such as the hair, the eyelashes and the eyebrows.

Volatile Oil

The at least one volatile oil (or organic solvents) may be chosen from hydrocarbon-based oils, silicone oils, and fluoro oils.

The at least one volatile oil may represent from 5% to 40%, such as from 7% to 20% by weight, for example from 8% to 15% by weight, relative to the total weight of the composition.

According to the present disclosure, the term “hydrocarbon-based oil” is understood to mean an oil mainly containing hydrogen and carbon atoms and possibly oxygen, nitrogen, sulfur or phosphorus atoms. The volatile hydrocarbon-based oils may be chosen from hydrocarbon-based oils containing from 8 to 16 carbon atoms, such as branched C₈-C₁₆ alkanes, for instance C₈-C₁₆ isoalkanes of petroleum origin (also known as isoparaffins), for instance isododecane (also known as 2,2,4,4,6-pentamethylheptane), isodecane and isohexadecane, for example the oils sold under the trade names Isopar® or Permethyl®, branched C₈-Q₁₆ esters such as isohexyl neopentanoate, and mixtures thereof. Other volatile hydrocarbon-based oils, for instance petroleum distillates, including those sold under the name Shell Solt® by the company Shell, may also be used.

In one embodiment, the hydrocarbon-based volatile oil(s) may be chosen from hydrocarbon-based volatile oils containing from 8 to 16 carbon atoms, such as isododecane, volatile silicone oils such as decamethylcyclopentasiloxane (D5) or dodecamethylcyclohexasiloxane (D6), and mixtures thereof.

Volatile silicone oils that may be used, by way of non-limiting example, include volatile linear or cyclic silicone oils, for instance those with a viscosity ≦6 centistokes (6×10⁻⁶ m²/s) and for example containing from 3 to 6 silicon atoms, these silicones optionally comprising at least one group chosen from alkyl and alkoxy groups containing 1 or 2 carbon atoms.

Examples of volatile silicone oils that may be used in the present disclosure, include but are not limited to octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, and dodecamethylpentasiloxane, and mixtures thereof.

According to at least one embodiment, the composition according to the present disclosure comprises at least one volatile silicone oil that is a volatile linear alkyltrisiloxane of general formula (I): 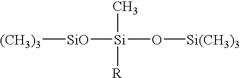
in which R represents an alkyl group containing from 2 to 4 carbon atoms, wherein at least one hydrogen atom of which may be replaced with a fluorine or chlorine atom.

Among the oils of general formula (I) that may be mentioned are:
3-butyl-1,1,1,3,5,5,5-heptamethyltrisiloxane,
3-propyl-1,1,1,3,5,5,5-heptamethyltrisiloxane, and
3-ethyl-1,1,1,3,5,5,5-heptamethyltrisiloxane,
   corresponding to the oils of formula (I) for which R is, respectively, a butyl group, a propyl group or an ethyl group.

The volatile linear alkyltrisiloxane oil of formula (I) may be prepared according to known processes for the synthesis of silicone compounds.

The oil of formula (I) for which R is an ethyl group is sold, for example, under the name Baysilone TP 3886 and the oil for which R is a butyl group is sold, for instance, under the name Baysilone TP 3887 by the company Bayer Silicones.

Volatile organic solvents, for example fluorinated solvents such as nonafluoromethoxybutane or perfluoromethylcyclopentane, may also be used.

According to at least one embodiment, the at least one volatile oil is present in an amount of greater than or equal to 5% by weight, for instance in an amount of at least 8% by weight and further for example in an amount of at least 10% by weight, relative to the total weight of the composition.

According to at least another embodiment, the at least one volatile oil may be chosen from isododecane, octamethyltrisiloxane, 3-ethyl-1,1,1,3,5,5,5-heptamethyltrisiloxane and octamethyltrisiloxane.

The composition according to the present disclosure may also comprise at least one non-volatile compound, which is water-insoluble and liquid at room temperature, such as at least one non-volatile organic solvent or oil, which may be chosen, for example, from non-volatile hydrocarbon-based oils and/or silicone oils and/or fluoro oils.

Non-volatile hydrocarbon-based oils that may be mentioned in a non-limiting manner include:

hydrocarbon-based oils of plant origin, such as triglycerides consisting of fatty acid esters of glycerol, the fatty acids of which may have varied chain lengths from C₄ to C₂₄, these chains possibly being linear or branched, and saturated or unsaturated; these oils may include wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy seed oil, pumpkin oil, sesame seed oil, marrow oil, rapeseed oil, blackcurrant oil, evening primrose oil, millet oil, barley oil, quinoa oil, rye oil, safflower oil, candlenut oil, passion flower oil and musk rose oil; or caprylic/capric acid triglycerides, for instance those sold by the company Stéarineries Dubois or those sold under the names Miglyol 810®, 812® and 818® by the company Dynamit Nobel,

synthetic ethers containing from 10 to 40 carbon atoms,

linear or branched hydrocarbons of mineral or synthetic origin, such as petroleum jelly, polydecenes, hydrogenated polyisobutene such as Parleam, squalane, and mixtures thereof,

synthetic esters, for instance oils of formula R₁COOR₂ in which R₁ represents a linear or branched fatty acid residue containing from 1 to 40 carbon atoms and R₂ represents a hydrocarbon-based chain, which can be, for instance, branched, containing from 1 to 40 carbon atoms, on the condition that R₁+R₂≧10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, alcohol or polyalcohol octanoates, decanoates or ricinoleates, for instance propylene glycol dioctanoate; hydroxylated esters, for instance isostearyl lactate or diisostearyl malate; and pentaerythritol esters,

fatty alcohols that are liquid at room temperature with a branched and/or unsaturated carbon-based chain containing from 12 to 26 carbon atoms, for instance octyidodecanol, isostearyl alcohol, oleyl alcohol, 2-hexyldecanol, 2-butyloctanol and 2-undecylpentadecanol,

higher fatty acids such as oleic acid, linoleic acid and linolenic acid, and mixtures thereof.

The non-volatile silicone oils that may be used in the composition according to the present disclosure may be non-volatile polydimethylsiloxanes (PDMS), polydimethylsiloxanes comprising alkyl or alkoxy groups, which are pendent and/or at the end of a silicone chain, these groups each containing from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyltrimethylsiloxysilicates.

The fluoro oils that may be used in the composition of the disclosure include, for example, fluorosilicone oils, fluoro polyethers and fluoro silicones as described in document EP-A-0 847 752.

The amount of non-volatile organic solvent or oil in a composition according to the disclosure may range from 0.01% to 20% by weight, for instance from 0.1% to 15% by weight and for example from 0.1% to 5% by weight, relative to the total weight of the composition.

Continuous Aqueous Phase

The continuous aqueous phase of the composition according to the disclosure comprises water and/or at least one water-soluble solvent.

In the present disclosure, the term “water-soluble solvent” is understood to mean a compound that is liquid at room temperature and water-miscible (miscibility in water of greater than 50% by weight at 25° C. and atmospheric pressure).

The water-soluble solvents that may be used in the compositions according to the disclosure may also be volatile.

Among the water-soluble solvents that may be used in the compositions according to the present disclosure, non-limiting mention may be made of lower monoalcohols containing from 1 to 5 carbon atoms, such as ethanol and isopropanol; glycols containing from 2 to 8 carbon atoms, such as ethylene glycol, propylene glycol, 1,3-butylene glycol and dipropylene glycol; C₃ and C₄ ketones and C₂-C₄ aldehydes.

The aqueous phase (water and optionally the water-miscible solvent) can be present in an amount ranging from 5% to 95% by weight, for instance ranging from 10% to 80% by weight and for example ranging from 15% to 60% by weight, relative to the total weight of the composition.

In at least one embodiment, the aqueous phase is present in an amount of at least 20% by weight, such as at least 30% and further for example at least 40% by weight, relative to the total weight of the composition.

Emulsifying System

The composition according to the present disclosure may contain emulsifying surfactants present in an amount ranging from 0.1% to 30%, for example from 1% to 15%, such as from 2% to 10% by weight, relative to the total weight of the composition.

According to the present disclosure an emulsifier appropriately chosen to obtain an oil-in-water emulsion is generally used. For instance, an emulsifier having at 25° C. an HLB (hydrophilic-lipophilic balance), according to Griffin, of greater than or equal to 8 may be used.

The HLB value according to Griffin is defined in J. Soc. Cosm. Chem. 1954 (Volume 5), pages 249-256.

These surfactants may be chosen from nonionic, anionic, cationic and amphoteric surfactants or emulsifying surfactants. Reference may be made to the document “Encyclopedia of Chemical Technology, Kirk-Othmer”, Volume 22, pages 333-432, 3^{rd} edition, 1979, Wiley, for the definition of the properties and (emulsifying) functions of surfactants, for example described on pages 347-377 of this reference, for anionic, amphoteric and nonionic surfactants.

For example, the surfactants that may be used in the composition according to the present disclosure can be chosen from:

a) nonionic surfactants with an HLB of greater than or equal to 8 at 25° C., used alone or as a mixture; non-limiting mention may be made of:

oxyethylenated and/or oxypropylenated ethers (which may comprise from 1 to 150 oxyethylene and/or oxypropylene groups) of glycerol;

oxyethylenated and/or oxypropylenated ethers (which may comprise from 1 to 150 oxyethylene and/or oxypropylene groups) of fatty alcohols (for instance a C₈-C₂₄ or a C₁₂-C₁₈ alcohol), such as oxyethylenated cetearyl alcohol ether containing 30 oxyethylene groups (CTFA name Ceteareth-30) and the oxyethylenated ether of the mixture of C₁₂-C₁₅ fatty alcohols comprising 7 oxyethylene groups (CTFA name C₁₂-C₁₅ Pareth-7 sold under the name Neodol 25-7® by Shell Chemicals);

fatty acid esters (such as a C₈-C₂₄ acid, and for example a C₁₆-C₂₂ acid) of polyethylene glycol (which may comprise from 1 to 150 ethylene glycol units), such as PEG-50 stearate and PEG-40 monostearate sold under the name Myrj 52P® by the company ICI Uniqema;

fatty acid esters (such as a C₈-C₂₄ acid, and for example a C₁₆-C₂₂ acid) of oxyethylenated and/or oxypropylenated glyceryl ethers (which may comprise from 1 to 150 oxyethylene and/or oxypropylene groups), for instance PEG-200 glyceryl monostearate sold under the name Simulsol 220 TM® by the company SEPPIC; glyceryl stearate polyethoxylated with 30 ethylene oxide groups, for instance the product Tagat S® sold by the company Goldschmidt, glyceryl oleate polyethoxylated with 30 ethylene oxide groups, for instance the product Tagat O® sold by the company Goldschmidt, glyceryl cocoate polyethoxylated with 30 ethylene oxide groups, for instance the product Varionic LI 13® sold by the company Sherex, glyceryl isostearate polyethoxylated with 30 ethylene oxide groups, for instance the product Tagat L® sold by the company Goldschmidt, and glyceryl laurate polyethoxylated with 30 ethylene oxide groups, for instance the product Tagat I® from the company Goldschmidt;

fatty acid esters (such as a C₈-C₂₄ acid, and for example a C₁₆-C₂₂ acid) of oxyethylenated and/or oxypropylenated sorbitol ethers (which may comprise from 1 to 150 oxyethylene and/or oxypropylene groups), for instance polysorbate 60 sold under the name Tween 60® by the company Uniqema;

dimethicone copolyol, such as the product sold under the name Q2-5220® by the company Dow Corning;

dimethicone copolyol benzoate (Finsolv SLB 101® and 201® from the company Finetex);

copolymers of propylene oxide and of ethylene oxide, also known as EO/PO polycondensates;

and mixtures thereof.

The EO/PO polycondensates include copolymers consisting of polyethylene glycol and polypropylene glycol blocks, for instance polyethylene glycol/polypropylene glycol/polyethylene glycol triblock polycondensates. These triblock polycondensates can have, for example, the following chemical structure:
H—(O—CH₂—CH₂)ₐ—(O—CH(CH₃)—CH₂)_{b}—(O—CH₂—CH₂)ₐ—OH,
wherein a ranges from 2 to 120 and b ranges from 1 to 100.

The EO/PO polycondensate may have a weight-average molecular weight ranging from 1,000 to 15,000 and further ranging from 2,000 to 13,000. The EO/PO polycondensate, for example, has a cloud point, at 10 g/l in distilled water, of greater than or equal to 20° C. and further for example greater than or equal to 60° C. The cloud point is measured according to ISO standard 1065. EO/PO polycondensates that may be used according to the present disclosure include but are not limited to polyethylene glycol/polypropylene glycol/polyethylene glycol triblock polycondensates sold under the name Synperonic®, for instance Synperonic PE/L44® and Synperonic PE/F127®, by the company ICI.

b) nonionic surfactants with an HLB of less than 8 at 25° C., optionally combined with at least one nonionic surfactant with an HLB of greater than 8 at 25° C., such as those mentioned above, such as:

saccharide esters and ethers, such as sucrose stearate, sucrose cocoate and sorbitan stearate, and mixtures thereof, for instance Arlatone 2121® sold by the company ICI;

fatty acid esters (such as a C₈-C₂₄ acid, and further for example a C₁₆-C₂₂ acid) of polyols, for example those of glycerol or of sorbitol, such as glyceryl stearate, glyceryl stearate such as the product sold under the name Tegin M® by the company Goldschmidt, glyceryl laurate such as the product sold under the name Imwitor 312® by the company Hüls, polyglyceryl-2 stearate, sorbitan tristearate or glyceryl ricinoleate;

the mixture of cyclomethicone/dimethicone copolyol sold under the name of Q2-3225C® by the company Dow Corning.

c) anionic surfactants such as:

C₁₆-C₃₀ fatty acid salts, for example those derived from amines, for instance triethanolamine stearate;

polyoxyethylenated fatty acid salts, for example those derived from amines or alkali metal salts, and mixtures thereof;

phosphoric esters and salts thereof, such as DEA oleth-10 phosphate (Crodafos N 10N from the company Croda) or monocetyl monopotassium phosphate (Amphisol K from Givaudan);

sulfosuccinates such as Disodium PEG-5 citrate lauryl sulfosuccinate and Disodium ricinoleamido MEA sulfosuccinate;

alkyl ether sulfates, such as sodium lauryl ether sulfate;

isethionates;

acylglutamates such as Disodium hydrogenated tallow glutamate (Amisoft HS-21 R® sold by the company Ajinomoto), and mixtures thereof.

Triethanolamine stearate is also suitable for the present disclosure. This surfactant is generally obtained by simple mixing of stearic acid and triethanolamine.

The compositions according to the disclosure may also contain at least one amphoteric surfactant, for instance N-acylamino acids such as N-alkylaminoacetates and disodium cocoamphodiacetate, and amine oxides such as stearamine oxide, or alternatively silicone surfactants, for instance dimethicone copolyol phosphates such as the product sold under the name Pecosil PS 100® by the company Phoenix Chemical.

Water-Soluble Gelling Agent

The composition according to the present disclosure may comprise at least one hydrophilic gelling agent.

The hydrophilic gelling agents that may be used in the compositions according to the disclosure may be chosen from:

homopolymers or copolymers of acrylic or methacrylic acid or the salts and esters thereof, for instance the products sold under the names Versicol F® or Versicol K® by the company Allied Colloid, Ultrahold 8® by the company Ciba-Geigy, and the polyacrylic acids of Synthalen K type;

copolymers of acrylic acid and of acrylamide sold in the form of the sodium salt thereof under the name Reten® by the company Hercules, sodium polymethacrylate sold under the name Darvan 7® by the company Vanderbilt, and the sodium salts of polyhydroxycarboxylic acids sold under the name Hydagen F® by the company Henkel;

polyacrylic acid/alkyl acrylate copolymers of the Pemulen type;

AMPS (polyacrylamidomethylpropanesulfonic acid partially neutralized with ammonia and highly crosslinked) sold by the company Clariant;

AMPS/acrylamide copolymers of the Sepigel® or Simulgel® type, sold by the company SEPPIC, and

AMPS/polyoxyethylenated alkyl methacrylate copolymers (crosslinked or non-crosslinked), and mixtures thereof.

The water-soluble film-forming polymers mentioned above may also act as hydrophilic gelling agent.

According to the present disclosure, the hydrophilic gelling agent may be present in the composition in a solids amount ranging from 0.01% to 60% by weight, such as from 0.5% to 40% by weight, for example from 1% to 30% by weight or further still from 5% to 20% by weight, relative to the total weight of the composition.

Structuring Agent

The composition according to the present disclosure may comprise at least one agent for structuring the oily phase or organic solvent (formed from the volatile or non-volatile organic solvents or oils described above), chosen from waxes, semi-crystalline polymers and lipophilic gelling agents.

The structuring agent may be present in an amount ranging from 5% to 80% by weight, for instance from 7% to 75% and even further, for example from 10% to 55% by weight, relative to the total weight of the composition.

The amount of oily structuring agent may be adjusted by a person skilled in the art as a function of the structuring properties of the said agents.

Wax(es)

The at least one wax that may be used in the context of the present disclosure is generally a lipophilic compound that is solid at room temperature (25° C.), with a solid/liquid reversible change of state, having a melting point of greater than or equal to 30° C., which may be up to 200° C. for example having a melting point up to 120° C.

By bringing the wax to the liquid form (melting), it is possible to make it miscible with oils and to form a microscopically uniform mixture, but upon cooling the mixture to room temperature, recrystallization of the wax in the oils of the mixture is obtained.

According to at least one embodiment of the present disclosure, the waxes that are suitable for use may have a melting point of greater than or equal to 45° C. and for example greater than or equal to 55° C.

For the purposes of the present disclosure, the melting point corresponds to the temperature of the most endothermic peak observed by thermal analysis (DSC) as described in ISO standard 11357-3; 1999. The melting point of the wax may be measured using a differential scanning calorimeter (DSC), for example the calorimeter sold under the name MDSC 2920 by the company TA Instruments.

The measuring protocol is as follows:

A sample of 5 mg of wax placed in a crucible is subjected to a first temperature rise ranging from −20° C. to 100° C., at a heating rate of 10° C. per minute, it is then cooled from 100° C. to −20° C. at a cooling rate of 10° C. per minute and is finally subjected to a second temperature increase ranging from −20° C. to 100° C. at a heating rate of 5° C. per minute. During the second temperature increase, the variation of the difference in power absorbed by the empty crucible and by the crucible containing the sample of wax is measured as a function of the temperature. The melting point of the compound is the temperature value corresponding to the top of the peak of the curve representing the variation in the difference in absorbed power as a function of the temperature.

The waxes that may be used in the compositions according to the present disclosure may be chosen from waxes that are solid at room temperature of animal, plant, mineral or synthetic origin, and mixtures thereof.

The waxes that may be used in the compositions according to the present disclosure generally have a hardness ranging from 0.01 MPa to 15 MPa, for instance greater than or equal to 0.05 MPa and further for example greater than or equal to 0.1 MPa.

The hardness is determined by measuring the compression force, measured at 20° C. using the texturometer sold under the name TA-XT2 by the company Rheo, equipped with a stainless-steel cylindrical spindle 2 mm in diameter, travelling at a measuring speed of 0.1 mm/second, and penetrating the wax to a penetration depth of 0.3 mm.

The measuring protocol is as follows:

The wax is melted at a temperature equal to the melting point of the wax +10° C. The molten wax is poured into a container 25 mm in diameter and 20 mm deep. The wax is recrystallized at room temperature (25° C.) for 24 hours such that the surface of the wax is flat and smooth, and the wax is then stored for at least 1 hour at 20° C. before measuring the hardness or the tack.

The texturometer spindle is displaced at a speed of 0.1 mm/s then penetrates the wax to a penetration depth of 0.3 mm. When the spindle has penetrated the wax to a depth of 0.3 mm, the spindle is held still for 1 second (corresponding to the relaxation time) and is then withdrawn at a speed of 0.5 mm/s.

The hardness value is the maximum compression force measured divided by the area of the texturometer cylinder in contact with the wax.

As examples of waxes that are suitable for the present disclosure, non-limiting mention may be made of hydrocarbon-based waxes, for instance beeswax, lanolin wax and Chinese insect waxes; rice bran wax, carnauba wax, candelilla wax, ouricury wax, alfalfa wax, berry wax, shellac wax, Japan wax and sumach wax; montan wax, orange wax, lemon wax, microcrystalline waxes, paraffins and ozokerite; polyethylene waxes, the waxes obtained by Fischer-Tropsch synthesis and waxy copolymers, and also esters thereof.

Non-limiting mention may also be made of waxes obtained by catalytic hydrogenation of animal or plant oils containing linear or branched C₈-C₃₂ fatty chains. Among these waxes that may be mentioned include but are not limited to isomerized jojoba oil such as the trans-isomerized partially hydrogenated jojoba oil manufactured or sold by the company Desert Whale under the commercial reference Iso-Jojoba-50, hydrogenated sunflower oil, hydrogenated castor oil, hydrogenated coconut oil, hydrogenated lanolin oil and bis(1,1,1-trimethylolpropane) tetrastearate sold under the name Hest 2T-4S® by the company Heterene.

Mention may also be made of silicone waxes and fluoro waxes.

The waxes obtained by hydrogenation of castor oil esterified with cetyl alcohol, sold under the names Phytowax ricin 16L64® and 22L73® by the company Sophim, may also be used. Such waxes are described in the document FR-A-2 792 190.

According to at least one embodiment of the present disclosure, the compositions according to the disclosure may comprise at least one “tacky” wax, i.e. a wax with a tack of greater than or equal to 1.7 N.s and a hardness of less than or equal to 3.5 MPa.

The tacky wax used may have, for instance, a tack ranging from 0.1 N.s to 10 N.s, for example ranging from 0.1 N.s to 5 N.s, for instance ranging from 0.2 N.s to 5 N.s and even further ranging from 0.3 N.s to 2 N.s.

The tack of the wax is determined by measuring the change in the force (compression force) as a function of time, at 20° C., according to the protocol indicated above for the hardness.

During the 1-second relaxation time, the force (compression force) decreases greatly until it becomes zero, and then, during the withdrawal of the spindle, the force (stretching force) becomes negative and then rises again to the value 0. The tack corresponds to the integral of the curve of the force as a function of time for the part of the curve corresponding to negative values of the force. The tack value is expressed in N.s.

The tacky wax that may be used generally has a hardness of less than or equal to 3.5 MPa, for example ranging from 0.01 MPa to 3.5 MPa, and further for example ranging from 0.05 MPa to 3 MPa.

Tacky waxes that may be used include a C₂₀-C₄₀ alkyl (hydroxystearyloxy)stearate (the alkyl group containing from 20 to 40 carbon atoms), alone or as a mixture.

such a wax is sold, for example, under the names Kester Wax K 82 P®, and Kester Wax K 80 Pe by the company Koster Keunen.

In the present disclosure, waxes provided in the form of small particles having a diameter expressed as the mean “effective” volume diameter D[4.3] of about from 0.5 to 30 micrometres, for example from 1 to 20 micrometres and further for example from 5 to 10 micrometres, which are referred to hereinafter as “microwaxes”, may also be used.

The particle sizes may be measured by various techniques; mention may be made of light-scattering techniques (dynamic and static), Coulter counter methods, sedimentation rate measurements (related to the size via Stokes' law) and microscopy. These techniques make it possible to measure a particle diameter and, for some of them, a particle size distribution.

The sizes and size distributions of the particles in the compositions according to the present disclosure are measured by static light scattering using a commercial granulometer such as the MasterSizer 2000 from Malvern. The data are processed on the basis of the Mie scattering theory. This theory, which is exact for isotropic particles, makes it possible to determine an “effective” particle diameter in the case of non-spherical particles. This theory is described for example, in the publication by Van de Hulst, H. C., “Light Scattering by Small Particles,” Chapters 9 and 10, Wiley, New York, 1957.

The composition may be characterized by its mean “effective” diameter by volume D[4.3], defined in the following manner:

$$D [ 4.3 ] = \frac{{\sum }_{i} {V}_{i} \cdot {d}_{i}}{{\sum }_{i} {V}_{i}}$$

in which Vᵢ represents the volume of the particles with an effective diameter dᵢ. This parameter may be described in the technical documentation of the granulometer.

The measurements are performed at 25° C. on a dilute particle dispersion, obtained from the composition in the following manner: 1) dilution by a factor of 100 with water, 2) homogenization of the solution, 3) standing of the solution for 18 hours, 4) recovery of the whitish uniform supernatant.

The “effective” diameter is obtained by taking a refractive index of 1.33 for water and a mean refractive index of 1.42 for the particles.

As microwaxes that may be used in the compositions according to the present disclosure, non-limiting mention may be made of carnauba microwaxes, such as the product sold under the name MicroCare 350® by the company Micro Powders, synthetic microwaxes, such as the product sold under the name MicroEase 114S® by the company Micro Powders, microwaxes consisting of a mixture of carnauba wax and polyethylene wax, such as the products sold under the names Micro Care 300® and 310® by the company Micro Powders, microwaxes consisting of a mixture of carnauba wax and of synthetic wax, such as the product sold under the name Micro Care 325® by the company Micro Powders, polyethylene microwaxes, such as the products sold under the names Micropoly 200®, 220®, 220L® and 250S® by the company Micro Powders, and polytetrafluoroethylene micropowders such as the products sold under the names Microslip 519® and 519 L® by the company Micro Powders.

The composition according to the present disclosure may comprise an amount of waxes ranging from 5% to 70% by weight, relative to the total weight of the composition; for instance containing from 7% to 50% and further for example containing from 10% to 45% thereof.

Semi-Crystalline Polymers

According to the present disclosure, the term “polymer” is understood to mean compounds containing at least two repeating units, such as at least three repeating units and for example at least ten repeating units. According to the present disclosure, the term “semi-crystalline polymer” is understood to mean polymers comprising a crystallizable portion, a crystallizable side chain or a crystallizable block in the skeleton, and an amorphous portion in the skeleton and having a first-order reversible phase-change temperature, such as melting (solid-liquid transition). When the crystallizable portion is in the form of a crystallizable block of the polymer skeleton, the amorphous portion of the polymer is in the form of an amorphous block; in this case, the semi-crystalline polymer is a block copolymer, for example, of the diblock, triblock or multiblock type, comprising at least one crystallizable block and at least one amorphous block. According to the present disclosure, the term “block” is generally understood to mean at least five identical repeating units. The crystallizable block(s) is (are) of chemical nature different than that of the amorphous block(s).

The semi-crystalline polymer has a melting point of greater than or equal to 30° C. (for example ranging from 30° C. to 80° C.), for instance ranging from 30° C. to 60° C. This melting point is a first-order change of state temperature.

This melting point may be measured by any known method, for example using a differential scanning calorimeter (DSC).

The semi-crystalline polymer(s) which may be used according to the present disclosure can have a number-average molecular mass of greater than or equal to 1,000. For instance, the semi-crystalline polymer(s) of the composition of the present disclosure can have a number-average molecular mass (Mn) ranging from 2,000 to 800,000, such as from 3,000 to 500,000, for example ranging from 4,000 to 150,000, and for example ranging from 100,000 and further ranging from 4,000 to 99,000. The crystalline polymer(s) may have a number-average molecular mass of greater than 5,600, for example ranging from 5,700 to 99,000. For the purposes of the present disclosure, the term “crystallizable chain or block” is understood to mean a chain or block which, if it were alone, would reversibly change from the amorphous state to the crystalline state, depending on whether the system is above or below the melting point. For the purposes of the disclosure, a chain is a group of atoms, which is pendent or lateral relative to the polymer skeleton. A block is a group of atoms belonging to the skeleton, this group constituting one of the repeating units of the polymer. The “crystallizable side chain” may be, for example, a chain containing at least six carbon atoms.

The semi-crystalline polymer may be chosen from block copolymers comprising at least one crystallizable block and at least one amorphous block, and homopolymers and copolymers bearing at least one crystallizable side chain per repeating unit, and mixtures thereof.

Such polymers are described, for example, in document EP 1 396 259.

A. Semi-Crystalline Polymers Containing Crystallizable Side Chains

Non-limiting mention may be made of the semi-crystalline polymers containing crystallizable side chains defined in U.S. Pat. No. 5,156,911 and the document WO-A-01/19333. They are homopolymers or copolymers comprising from 50% to 100% by weight of units resulting from the polymerization of at least one monomer bearing a crystallizable hydrophobic side chain.

These homopolymers or copolymers may be of any nature, provided that they meet the conditions mentioned previously.

B. Polymers Bearing in the Skeleton at Least One Crystallizable Block

These polymers bearing at least one crystallizable block in the skeleton include but are not limited to block copolymers consisting of at least two blocks of different chemical nature, one of which is crystallizable.

For example, the block polymers defined in U.S. Pat. No. 5,156,911 may be used;

The block copolymers of olefin or of cycloolefin containing a crystallizable chain, for instance those derived from the block polymerization of:

cyclobutene, cyclohexene, cyclooctene, norbornene (i.e. bicyclo(2,2,1)-2-heptene), 5-methylnorbornene, 5-ethylnorbornene, 5,6-dimethylnorbornene, 5,5,6-trimethylnorbornene, 5-ethylidenenorbornene, 5-phenylnorbornene, 5-benzylnorbornene, 5-vinylnorbornene, 1,4,5,8-dimethano-1,2,3,4,4a,5,8a-octahydronaphthalene, dicyclopentadiene, or mixtures thereof,

with ethylene, propylene, 1-butene, 3-methyl-1-butene, 1-hexene, 4-methyl-1-pentene, 1-octene, 1-decene or 1-eicosene, or mixtures thereof,

and for instance, copoly(ethylene/norbornene) blocks and (ethylene/propylene/ethylidene-norbornene) block terpolymers. Those resulting from the block copolymerization of at least two C₂-C₁₆, for instance C₂-C₁₂ and for example C₄-C₁₂ α-olefins such as those mentioned above, including but not limited to block bipolymers of ethylene and of 1-octene may also be used.

The copolymers may be copolymers containing at least one crystallizable block, the rest of the copolymer being amorphous (at room temperature). These copolymers may also contain two crystallizable blocks of different chemical nature. The copolymers may include those that simultaneously contain at room temperature a crystallizable block and an amorphous block that are both hydrophobic and lipophilic, sequentially distributed; mention may be made, for example, of polymers containing one of the crystallizable blocks and one of the amorphous blocks below:

Blocks that are crystallizable by nature: a) of polyester type, for instance poly(alkylene terephthalate), b) of polyolefin type, for instance polyethylenes or polypropylenes.

Amorphous and lipophilic blocks, for instance: amorphous polyolefins or copoly(olefin)s such as poly(isobutylene), hydrogenated polybutadiene or hydrogenated poly(isoprene).

As examples of such copolymers containing a crystallizable block and an amorphous block, non-limiting mention may be made of:

a) poly(ε-caprolactone)-b-poly(butadiene) block copolymers, for example hydrogenated, such as those described in the article “Melting behavior of poly(ε-caprolactone)-block-polybutadiene copolymers” from S. Nojima, Macromolecules, 32, 3727-3734 (1999),

b) the hydrogenated block or multiblock poly(butylene terephthalate)-b-poly(isoprene) block copolymers cited in the article “Study of morphological and mechanical properties of PP/PBT” by B. Boutevin et al., Polymer Bulletin, 34, 117-123 (1995),

c) the poly(ethylene)-b-copoly(ethylene/propylene) block copolymers cited in the articles “Morphology of semi-crystalline block copolymers of ethylene-(ethylene-altpropylene)” by P. Rangarajan et al., Macromolecules, 26, 4640-4645 (1993) and “Polymer aggregates with crystalline cores: the system poly(ethylene)-poly(ethylene-propylene)” by P. Richter et al., Macromolecules, 30, 1053-1068 (1997),

d) the poly(ethylene)-b-poly(ethylethylene) block copolymers cited in the general article “Crystallization in block copolymers” by I. W. Hamley, Advances in Polymer Science, Vol. 148, 113-137 (1999).

The semi-crystalline polymers in the composition according to the present disclosure may be, for example, non-crosslinked.

According to at least one embodiment of the present disclosure, the polymer is chosen from copolymers resulting from the polymerization of at least one monomer containing a crystallizable chain chosen from saturated C₁₄-C₂₄ alkyl (meth)acrylates, C₁₁-C₁₅ perfluoroalkyl (meth)acrylates, C₁₄ to C₂₄ N-alkyl(meth)acrylamides with or without a fluorine atom, vinyl esters containing C₁₄ to C₂₄ alkyl or perfluoroalkyl chains, vinyl ethers containing C₁₄ to C₂₄ alkyl or perfluoralkyl chains, C₁₄ to C₂₄ α-olefins, para-alkylstyrenes with an alkyl group containing from 12 to 24 carbon atoms, with at least one optionally fluorinated C₁ to C₁₀ monocarboxylic acid ester or amide, which may be represented by the following formula:

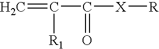

in which R₁ is chosen from H and CH₃, R is chosen from optionally fluorinated C₁-C₁₀ alkyl groups, and X is chosen from O, NH and NR₂, in which R₂ is chosen from optionally fluorinated C₁-C₁₀ alkyl groups. According to at least one embodiment of the present disclosure, the polymer may be derived from a monomer containing a crystallizable chain chosen from saturated C₁₄-C₂₂ alkyl (meth)acrylates.

Among examples of a semi-crystalline polymer that may be used in the composition according to the disclosure, non-limiting mention may be made of the Intelimer® products from the company Landec described in the brochure “Intelimer® Polymers”, Landec IP22 (Rev. 4-97). These polymers are in solid form at room temperature (25° C.). They bear crystallizable side chains and have the above formula X.

Lipophilic Gelling Agents

The gelling agents that may be used in the compositions according to the present disclosure may be organic or mineral, polymeric or molecular lipophilic gelling agents.

Mineral lipophilic gelling agents that may be mentioned include but are not limited to optionally modified clays, for instance hectorites modified with a C₁₀ to C₂₂ fatty acid ammonium chloride, for instance hectorite modified with distearyldimethylammonium chloride, for instance the product sold under the name Bentone 38V® by the company Elementis.

Non-limiting mention may also be made of fumed silica optionally subjected to a hydrophobic surface treatment, the particle size of which is less than 1 μm. For instance, it is possible to chemically modify the surface of the silica, by chemical reaction generating a reduced number of silanol groups present at the surface of the silica. It is possible, for example, to substitute silanol groups with hydrophobic groups: a hydrophobic silica is then obtained. The hydrophobic groups may be, by way of non-limiting example:

trimethylsiloxyl groups, which may be obtained by treating fumed silica in the presence of hexamethyldisilazane. Silicas thus treated are known as “silica silylate” according to the CTFA (6th edition, 1995). They are sold, for example, under the references Aerosil R812® by the company Degussa, and Cab-O-Sil TS-530® by the company Cabot;

dimethylsilyloxyl or polydimethylsiloxane groups, which may be obtained by treating fumed silica in the presence of polydimethylsiloxane or dimethyldichlorosilane. Silicas thus treated are known as “silica dimethyl silylate” according to the CTFA (6th edition, 1995). They are sold, for example, under the references Aerosil R972® and Aerosil R974® by the company Degussa, and Cab-O-Sil TS-610® and Cab-O-Sil TS-720® by the company Cabot.

The hydrophobic fumed silica may have a particle size that may be nanometric to micrometric, for example ranging from about 5 to 200 nm.

The polymeric organic lipophilic gelling agents may include, for example, partially or totally crosslinked elastomeric organopolysiloxanes of three-dimensional structure, for instance those sold under the names KSG6®, KSG16® and KSG18® from Shin-Etsu, Trefil E-505C® or Trefil E-506C® from Dow Corning, Gransil SR-CYC®, SR DMF 10®, SR-DC556®, SR 5CYC gel®, SR DMF 10 gel® and SR DC 556 gel® from Grant Industries and SF 1204® and JK 113® from General Electric; ethylcellulose, for instance the product sold under the name Ethocel by Dow Chemical; polycondensates of polyamide type resulting from condensation between (a) at least one acid chosen from dicarboxylic acids containing at least 32 carbon atoms, such as fatty acid dimers, and (b) an alkylenediamine, for example ethylenediamine, in which the polyamide polymer comprises at least one carboxylic acid end group esterified or amidated with at least one saturated and linear monoalcohol or one saturated and linear monoamine containing from 12 to 30 carbon atoms, and for example ethylenediamine/stearyl dilinoleate copolymers such as the product sold under the name Uniclear 100 VG® by the company Arizona Chemical; silicone polyamides of the polyorganosiloxane type, for instance those described in U.S. Pat. Nos. 5,874,069, 5,919,441, 6,051,216 and 5,981,680, for instance those sold under the reference Dow Corning 2-8179 Gellant by the company Dow Corning; galactomannans comprising from one to six, such as from two to four hydroxyl groups per saccharide, substituted with a saturated or unsaturated alkyl chain, for instance guar gum alkylated with C₁ to C₆, such as C₁ to C₃, alkyl chains, and mixtures thereof. Block copolymers of “diblock,” “riblock” or “radial” type, of the polystyrene/polyisoprene or polystyrene/polybutadiene type, such as the products sold under the name Luvitol HSB® by the company BASF, of the polystyrene/copoly(ethylene-propylene) type, such as the products sold under the name Kraton® by the company Shell Chemical Co., or of the polystyrene/copoly(ethylene-butylene) type, and mixtures of triblock and radial (star) copolymers in isododecane, such as those sold by the company Penreco under the name Versagel®, for instance the mixture of butylene/ethylene/styrene triblock copolymer and of ethylene/propylene/styrene star copolymer in isododecane (Versagel M 5960), may also be used.

Among the gelling agents that may be used in the compositions according to the present disclosure, non-limiting mention may also be made of fatty acid esters of dextrin, such as dextrin palmitates, for example the products sold under the name Rheopearl TL® or Rheopearl KL® by the company Chiba Flour.

Film-Forming Polymer

According to at least one embodiment, the composition according to the present disclosure may further comprise at least one film-forming polymer.

The at least one film-forming polymer may be present in the composition according to the disclosure in a solids (or active material) amount ranging from 0.1% to 30% by weight, for example ranging from 0.5% to 20% by weight and further for instance ranging from 1% to 15% by weight, relative to the total weight of the composition.

In the present disclosure, the expression “film-forming polymer” is understood to mean a polymer that is capable, by itself or in the presence of an auxiliary film-forming agent, of forming a macroscopically continuous film that adheres to the keratin fibers, such as a cohesive film and for instance a film whose cohesion and mechanical properties are such that the film may be isolated and manipulated separately, for example when the film is made by casting on a non-stick surface, for instance a Teflon-coated or silicone-coated surface.

Among the film-forming polymers that may be used in the composition of the present disclosure, non-limiting mention may be made of synthetic polymers, of free-radical type or of polycondensate type, and polymers of natural origin, and mixtures thereof.

According to the present disclosure, the expression “free-radical film-forming polymer” is understood to mean a polymer obtained by polymerization of unsaturated and ethylenically unsaturated monomers, each monomer being capable of homopolymerizing (unlike polycondensates).

The film-forming polymers of free-radical type may be, for example, vinyl polymers or copolymers, such as acrylic polymers.

The vinyl film-forming polymers may result from the polymerization of ethylenically unsaturated monomers containing at least one acidic group and/or esters of these acidic monomers and/or amides of these acidic monomers.

Monomers bearing an acidic group which may be used include but are not limited to α,β-ethylenic unsaturated carboxylic acids such as acrylic acid, methacrylic acid, crotonic acid, maleic acid or itaconic acid. In one embodiment of the present disclosure, for example, (Meth)acrylic acid and crotonic acid can be used, such as (meth)acrylic acid.

The esters of acidic monomers may be chosen from (meth)acrylic acid esters (also known as (meth)acrylates), such as (meth)acrylates of an alkyl, for example a C₁-C₃₀ alkyl, and further for instance a C₁-C₂₀ alkyl, (meth)acrylates of an aryl, for example a C₆-C₁₀ aryl, and (meth)acrylates of a hydroxyalkyl, such as a C₂-C₆ hydroxyalkyl.

Alkyl (meth)acrylates that may be mentioned include but are not limited to methyl methacrylate, ethyl methacrylate, butyl methacrylate, isobutyl methacrylate, 2-ethylhexyl methacrylate, lauryl methacrylate and cyclohexyl methacrylate.

Hydroxyalkyl (meth)acrylates that may be mentioned include but are not limited to hydroxyethyl acrylate, 2-hydroxypropyl acrylate, hydroxyethyl methacrylate and 2-hydroxypropyl methacrylate.

Aryl (meth)acrylates that may be mentioned include but are not limited to benzyl acrylate and phenyl acrylate.

The (meth)acrylic acid esters that may be used include but are not limited to the alkyl (meth)acrylates.

According to the present disclosure, the alkyl group of the esters may be either fluorinated or perfluorinated, i.e. some or all of the hydrogen atoms of the alkyl group are substituted with fluorine atoms.

Examples of amides of the acid monomers that may be mentioned include but are not limited to (meth)acrylamides, such as N-alkyl(meth)acrylamides, for example a C₂-C₁₂ alkyl. The N-alkyl(meth)acrylamides that may be mentioned include but are not limited to N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. For instance, these monomers may be polymerized with acid monomers and/or esters thereof and/or amides thereof, such as those mentioned above.

Examples of vinyl esters that may be mentioned include but are not limited to vinyl acetate, vinyl neodecanoate, vinyl pivalate, vinyl benzoate and vinyl t-butylbenzoate.

styrene monomers that may be mentioned include but are not limited to styrene and α-methylstyrene.

Among the film-forming polycondensates that may be used, non-limiting mention may be made of polyurethanes, polyesters, polyesteramides, polyamides, epoxyester resins and polyureas.

The polyurethanes may be chosen from anionic, cationic, nonionic and amphoteric polyurethanes, polyurethane-acrylics, polyurethane-polyvinylpyrrolidones, polyester-polyurethanes, polyether-polyurethanes, polyureas and polyurea/polyurethanes, and mixtures thereof.

The polyesters may be obtained, in a known manner, by polycondensation of dicarboxylic acids with polyols, for example diols.

The dicarboxylic acid may be aliphatic, alicyclic or aromatic. Examples of such acids that may be mentioned include but are not limited to: oxalic acid, malonic acid, dimethylmalonic acid, succinic acid, glutaric acid, adipic acid, pimelic acid, 2,2-dimethylglutaric acid, azeleic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbomanedicarboxylic acid, diglycolic acid, thiodipropionic acid, 2,5-naphthalenedicarboxylic acid or 2,6-naphthalenedicarboxylic acid. These dicarboxylic acid monomers may be used alone or as a combination of at least two dicarboxylic acid monomers. Among these monomers, in one embodiment of the present disclosure, for example, phthalic acid, isophthalic acid and terephthalic acid may be used.

The diol may be chosen from aliphatic, alicyclic and aromatic diols. For example, the diol used may be chosen from: ethylene glycol, diethylene glycol, triethylene glycol, 1,3-propanediol, cyclohexanedimethanol and 4-butanediol. Other polyols that may be used include but are not limited to glycerol, pentaerythritol, sorbitol and trimethylolpropane.

The polyesteramides may be obtained in a manner analogous to that of the polyesters, by polycondensation of diacids with diamines or amino alcohols. Diamines that may be used can include ethylenediamine, hexamethylenediamine and meta- or para-phenylenediamine. An amino alcohol that may be used includes monoethanolamine.

The polyester may also comprise at least one monomer bearing at least one group —SO₃M, with M representing a hydrogen atom, an ammonium ion NH₄⁺ or a metal ion such as, for example, an Na⁺, Li⁺, K⁺, Mg²⁺, Ca²⁺, Cu²⁺, Fe²⁺ or Fe³⁺ ion. A difunctional aromatic monomer comprising such a group —SO₃M may also be used.

The aromatic nucleus of the difunctional aromatic monomer also bearing a group —SO₃M as described above may be chosen, for example, from benzene, naphthalene, anthracene, biphenyl, oxybiphenyl, sulfonylbiphenyl and methylenebiphenyl nuclei. As examples of difunctional aromatic monomers also bearing a group —SO₃M, non-limiting mention may be made of: sulfoisophthalic acid, sulfoterephthalic acid, sulfophthalic acid, and 4-sulfonaphthalene-2,7-dicarboxylic acid.

The copolymers that may be used include those based on isophthalate/sulfoisophthalate, for example copolymers obtained by condensation of diethylene glycol, cyclohexanedimethanol, isophthalic acid and sulfoisophthalic acid.

The polymers of natural origin, optionally modified, may be chosen from shellac resin, sandarac gum, dammar resins, elemi gums, copal resins and cellulose polymers, and mixtures thereof.

According to at least one embodiment of the composition according to the disclosure, the film-forming polymer may be a water-soluble polymer and may be present in the aqueous phase of the composition; the polymer may be solubilized in the aqueous phase of the composition. Examples of water-soluble film-forming polymers that may be mentioned include but are not limited to:

proteins, for instance proteins of plant origin such as wheat proteins and soybean proteins; proteins of animal origin such as keratins, for example keratin hydrolyzates and sulfonic keratins;

polymers of cellulose such as hydroxyethylcellulose, hydroxypropylcellulose, methylcellulose, ethylhydroxyethylcellulose and carboxymethylcellulose, and quaternized cellulose derivatives;

acrylic polymers or copolymers, such as polyacrylates or polymethacrylates;

vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol;

polymers of natural origin, which are optionally modified, such as:

gum arabics, guar gum, xanthan derivatives, karaya gum;

alginates and carrageenans;

glycosaminoglycans, hyaluronic acid and derivatives thereof;

shellac resin, sandarac gum, dammar resins, elemi gums and copal resins;

deoxyribonucleic acid;

mucopolysaccharides such as chondroitin sulfate,

and mixtures thereof.

According to at least one embodiment of the present disclosure, the film-forming polymer may be a polymer dissolved in a liquid fatty phase comprising organic solvents or oils such as those described above (the film-forming polymer is thus said to be a liposoluble polymer). For the purposes of the present disclosure, the expression “liquid fatty phase” is understood to mean a fatty phase which is liquid at room temperature (25° C.) and atmospheric pressure (760 mmHg, i.e. 10⁵ Pa), composed of at least one fatty substance that is liquid at room temperature, also known as oils, which are generally mutually compatible.

The liquid fatty phase may comprise a volatile oil, optionally mixed with a non-volatile oil, the oils possibly being chosen from those mentioned above.

Examples of liposoluble polymers which may be mentioned include but are not limited to copolymers of vinyl ester (the vinyl group being directly linked to the oxygen atom of the ester group and the vinyl ester containing a saturated, linear or branched hydrocarbon-based radical of 1 to 19 carbon atoms, linked to the carbonyl of the ester group) and of at least one other monomer which may be a vinyl ester (other than the vinyl ester already present), an α-olefin (containing from 8 to 28 carbon atoms), an alkyl vinyl ether (in which the alkyl group comprises from 2 to 18 carbon atoms) or an allylic or methallylic ester (containing a saturated, linear or branched hydrocarbon-based radical of 1 to 19 carbon atoms, linked to the carbonyl of the ester group).

These copolymers may be crosslinked with the aid of crosslinking agents, which may be either of the vinyl type or of the allylic or methallylic type, such as tetraallyloxyethane, divinylbenzene, divinyl octanedioate, divinyl dodecanedioate and divinyl octadecanedioate.

Examples of these copolymers that may be mentioned include but are not limited to the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene; vinyl dimethylpropionate/vinyl laurate, crosslinked with 0.2% divinylbenzene; vinyl acetate/octadecyl vinyl ether, crosslinked with 0.2% tetraallyloxyethane; vinyl acetate/allyl stearate, crosslinked with 0.2% divinylbenzene; vinyl acetate/1-octadecene, crosslinked with 0.2% divinylbenzene; and allyl propionate/allyl stearate, crosslinked with 0.2% divinylbenzene.

Examples of liposoluble film-forming polymers which may also be mentioned include but are not limited to liposoluble copolymers, for example those resulting from the copolymerization of vinyl esters containing from 9 to 22 carbon atoms or of alkyl acrylates or methacrylates, and alkyl radicals containing from 10 to 20 carbon atoms.

such liposoluble copolymers may be chosen from polyvinyl stearate, polyvinyl stearate crosslinked with the aid of divinylbenzene, of diallyl ether or of diallyl phthalate, polystearyl (meth)acrylate, polyvinyl laurate and polylauryl (meth)acrylate, it being possible for these poly(meth)acrylates to be crosslinked with the aid of ethylene glycol dimethacrylate or tetraethylene glycol dimethacrylate.

The liposoluble copolymers defined above are known and are described for example in the document FR-A-2 232 303; they may have a weight-average molecular weight ranging from 2,000 to 500,000 for example ranging from 4,000 to 200,000.

As liposoluble film-forming polymers which may be used in the present disclosure, non-limiting mention may also be made of polyalkylenes for instance copolymers of C₂-C₂₀ alkenes, such as polybutene, alkylcelluloses with a linear or branched, saturated or unsaturated C₁-C₈ alkyl radical, for instance ethylcellulose and propylcellulose, copolymers of vinylpyrrolidone (VP) such as copolymers of vinylpyrrolidone and of C₂ to C₄₀ and further for example C₃ to C₂₀ alkene. As examples of VP copolymers which may be used in the present disclosure, non-limiting mention may be made of the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

Non-limiting mention may also be made of silicone resins, which are generally soluble or swellable in silicone oils, which are crosslinked polyorganosiloxane polymers. The nomenclature of silicone resins is known under the name “MDTQ”, the resin being described as a function of the various siloxane monomer units it comprises, each of the letters “MDTQ” characterizing a type of unit.

Examples of commercially available polymethylsilsesquioxane resins that may be mentioned, in a non-limiting manner, include those sold:

by the company Wacker under the reference Resin MK, such as Belsil PMS MK; and

by the company Shin-Etsu under the reference KR-220L.

siloxysilicate resins that may be mentioned include but are not limited to trimethyl siloxysilicate (TMS) resins such as those sold under the reference SR 1000 by the company General Electric or under the reference TMS 803 by the company Wacker. Non-limiting mention may also be made of the trimethyl siloxysilicate resins sold in a solvent such as cyclomethicone, sold under the name KF-7312J by the company Shin-Etsu, and DC 749 and DC 593 by the company Dow Corning.

Non-limiting mention may further be made of silicone resin copolymers such as those mentioned above with polydimethylsiloxanes, for instance the pressure-sensitive adhesive copolymers sold by the company Dow Corning under the reference Bio-PSA and described U.S. Pat. No. 5,162,410, or the silicone copolymers derived from the reaction of a silicone resin, such as those described above, and of a diorganosiloxane, as described in document WO 2004/073 626.

According to at least one embodiment of the present disclosure, the film-forming polymer is a film-forming linear block ethylenic polymer, which may comprise at least a first block and at least a second block with different glass transition temperatures (Tg), the said first and second blocks being linked together via an intermediate block comprising at least one constituent monomer of the first block and at least one constituent monomer of the second block.

The first and second blocks of the block polymer may be mutually incompatible.

such polymers are described, for example, in the documents EP 1 411 069 or WO 04/028 488.

According to at least one embodiment, the composition according to the disclosure can have a liposoluble or lipodispersible film-forming polymer solids amount of less than 5% by weight, for example less than or equal to 4% by weight and further for example less than or equal to 3% by weight, relative to the total weight of the composition, and further for example the composition can be free of liposoluble or lipodispersible film-forming polymer.

Accordingly, another aspect of the present disclosure is a keratin fiber coating composition comprising a continuous aqueous phase, at least one volatile oil, and at least one liposoluble or lipodispersible film-forming polymer in a polymer solids amount of less than or equal to 5% by weight, relative to the total weight of the composition, wherein when the composition forms a film on the keratin fiber, the film has a water resistance of greater than or equal to −4.5.

Another aspect of the disclosure is also a keratin fiber coating composition comprising a continuous aqueous phase, at least one volatile oil, and at least one liposoluble or lipodispersible film-forming polymer in a polymer solids amount of less than or equal to 5% by weight, relative to the total weight of the composition, wherein when the composition forms a film on the keratin fiber, the film has a sebum resistance such that ΔL is greater than or equal to −2.5.

The present disclosure also relates to the use of at least one volatile oil, and of at least one liposoluble or lipodispersible film-forming polymer in a polymer solids amount of less than or equal to 5% by weight relative to the total weight of the composition, in a keratin fiber coating composition comprising a continuous aqueous phase, to obtain a composition that, when it forms a film deposited on the keratin fibers, it has a water resistance of greater than or equal to −4.5 and/or a sebum resistance such that ΔL is greater than or equal to −2.5.

The film-forming polymer may also be present in the composition in the form of particles dispersed in an aqueous phase or in a non-aqueous solvent phase, which is generally known as a latex or pseudolatex. The techniques for preparing these dispersions are well known to those skilled in the art.

Aqueous dispersions of film-forming polymers that may be used include, by way of non-limiting example, the acrylic dispersions sold under the names Neocryl XK-90®, Neocryl A-1070®, Neocryl A-1090®, Neocryl BT-62®, Neocryl A-1079® and Neocryl A-523® by the company Avecia-Neoresins, Dow Latex 432® by the company Dow Chemical, Daitosol 5000 AD® or Daitosol 5000 SJ® by the company Daito Kasey Kogyo; Syntran 5760® by the company Interpolymer, or the aqueous dispersions of polyurethane sold under the names Neorez R-981® and Neorez R-974® by the company Avecia-Neoresins, Avalure UR-405®, Avalure UR-410®, Avalure UR-425®, Avalure UR-450®, Sancure 875®, Sancure 861®, Sancure 878® and Sancure 2060® by the company Goodrich, Impranil 85® by the company Bayer and Aquamere H-1511® by the company Hydromer; the sulfopolyesters sold under the brand name Eastman AQ® by the company Eastman Chemical Products, and vinyl dispersions, for instance Mexomer PAM® from the company Chimex, and mixtures thereof.

Non-limiting examples of non-aqueous film-forming polymer dispersions that may also be mentioned include acrylic dispersions in isododecane, for instance Mexomer PAP®from the company Chimex, and dispersions of particles of a grafted ethylenic polymer, for example an acrylic polymer, in a liquid fatty phase, wherein the ethylenic polymer, for example, may be dispersed in the absence of additional stabilizer at the surface of the particles as described in document WO 04/055 081.

The composition according to the present disclosure may further comprise at least one plasticizer that promotes the formation of a film with the film-forming polymer. Such a plasticizer may be chosen from any compound known to those skilled in the art as being capable of fulfilling the desired function.

Dyestuff

The composition according to the present disclosure may also comprise at least one dyestuff, for instance, chosen from pulverulent dyes, liposoluble dyes and water-soluble dyes.

The pulveru lent dyestuffs may be chosen from pigments and nacres.

The pigments may be white or colored, mineral and/or organic, and coated or uncoated. Among the mineral pigments that may be mentioned, non-limiting examples include titanium dioxide, optionally surface-treated, zirconium oxide, zinc oxide or cerium oxide, and also iron oxide or chromium oxide, manganese violet, ultramarine blue, chromium hydrate and ferric blue. Tthe organic pigments that may be mentioned in a non-limiting manner, include carbon black, pigments of D & C type, and lakes based on cochineal carmine or on barium, strontium, calcium or aluminium.

The nacres may be chosen from white nacreous pigments such as mica coated with titanium or with bismuth oxychloride, colored nacreous pigments such as titanium mica with iron oxides, titanium mica with, for instance, ferric blue or chromium oxide, titanium mica with an organic pigment of the abovementioned type, and also nacreous pigments based on bismuth oxychloride.

The liposoluble dyes can be, for example, Sudan Red, D&C Red 17, D&C Green 6, β-carotene, soybean oil, Sudan Brown, D&C Yellow 11, D&C Violet 2, D&C Orange 5, quinoline yellow and annatto.

The at least one dyestuff may be present in an amount ranging from 0.01% to 30% by weight, relative to the total weight of the composition.

Fillers

The composition according to the present disclosure may also comprise at least one filler.

The fillers may be chosen from those that are well known to persons skilled in the art and commonly used in cosmetic compositions. The fillers may be mineral or organic, and lamellar or spherical. Non-limiting mention may be made of talc, mica, silica, kaolin, polyamide powders, for instance the Nylon® sold under the trade name Orgasol® by the company Atochem, poly-β-alanine powders and polyethylene powders, powders of tetrafluoroethylene polymers, for instance Teflon®, lauroyllysine, starch, boron nitride, expanded polymeric hollow microspheres such as those of polyvinylidene chloride/acrylonitrile, for instance the products sold under the name Expancel® by the company Nobel Industrie, acrylic powders, such as those sold under the name Polytrap® by the company Dow Corning, polymethyl methacrylate particles and silicone resin microbeads (for example Tospearls® from Toshiba), precipitated calcium carbonate, magnesium carbonate and magnesium hydrocarbonate, hydroxyapatite, hollow silica microspheres (Silica Beads® from Maprecos), glass or ceramic microcapsules, metal soaps derived from organic carboxylic acids containing from 8 to 22 carbon atoms such as for example from 12 to 18 carbon atoms, for example zinc, magnesium or lithium stearate, zinc laurate and magnesium myristate.

It is also possible to use a compound that is capable of swelling on heating, for instance heat-expandable particles such as non-expanded microspheres of copolymer of vinylidene chloride/acrylonitrile/methyl methacrylate or of acrylonitrile homopolymer copolymer, for instance those sold, respectively, under the references Expancel® 820 DU 40 and Expancel® 007WU by the company Akzo Nobel.

The at least one filler may be present in an amount ranging from 0.1% to 25%, for instance from 1% to 20% by weight, relative to the total weight of the composition.

The composition of the present disclosure may also comprise at least one adjuvant usually used in cosmetics, such as antioxidants, preserving agents, fibers, fragrances, neutralizers, gelling agents, thickeners, vitamins, coalescers and plasticizers, and mixtures thereof.

Fibers

The composition according to the present disclosure may also comprise fibers to allow an improvement in the lengthening effect.

According to the present disclosure, the term “fiber” should be understood as meaning an object of length L and diameter D such that L is very much greater than D, D being the diameter of the circle in which the cross section of the fiber is inscribed. In particular, the ratio L/D (or shape factor) is chosen in the range from 3.5 to 2,500, such as from 5 to 500 and for example from 5 to 150.

The fibers that may be used in the composition of the disclosure may be mineral or organic fibers of synthetic or natural origin. They may be short or long, individual or organized, for example braided, and hollow or solid. They may have any shape, and may, for instance, have a circular or polygonal (square, hexagonal or octagonal) cross section, depending on the intended specific application. For example, in one embodiment of the present disclosure, the fibers' ends are blunt and/or polished to prevent injury.

For instance, the fibers can have a length ranging from 1 μm to 10 mm, such as from 0.1 mm to 5 mm and for example from 0.3 mm to 3.5 mm. Their cross section may be within a circle of diameter ranging from 2 nm to 500 μm, possibly ranging from 100 nm to 100 μm and for example from 1 μm to 50 μm. The weight or yarn count of the fibers is often given in denier or decitex, and represents the weight in grams per 9 km of yarn. According to one aspect of the disclosure, the fibers may have a yarn count chosen in the range from 0.15 to 30 denier, for example from 0.18 to 18 denier.

The fibers that may be used in the composition of the present disclosure may be chosen from rigid or non-rigid fibers, and may be of synthetic or natural, mineral or organic origin.

Moreover, the fibers may or may not be surface-treated, may be coated or uncoated, and may be colored or uncolored.

As fibers that may be used in the composition according to the present disclosure, non-limiting mention may be made of non-rigid fibers such as polyamide (Nylon®) fibers or rigid fibers such as polyimideamide fibers, for instance those sold under the names Kermel® and Kermel Tech® by the company Rhodia or poly(p-phenyleneterephthalamide) (or aramid) fibers sold for example under the name Kevlar® by the company DuPont de Nemours.

The fibers may be present in the composition according to the present disclosure in an amount ranging from 0.01% to 10% by weight, for example ranging from 0.1% to 5% by weight and further for example ranging from 0.3% to 3% by weight, relative to the total weight of the composition.

Cosmetic Active Agents

The compositions of the present disclosure may further comprise at least one cosmetic active agent. As cosmetic active agents that may be used in the compositions according to the present disclosure, non-limiting mention may be made of, for example, antioxidants, preserving agents, fragrances, neutralizers, emollients, moisturizers, vitamins and screening agents, for example sunscreens.

Needless to say, a person skilled in the art will take care to select the optional additional additives and/or the amount thereof such that the beneficial properties of the composition according to the disclosure are not, or are not substantially, adversely affected by the envisaged addition.

The compositions according to the present disclosure may be prepared according to methods known to those skilled in the art.

The composition according to the disclosure may be packaged in a container delimiting at least one compartment that comprises the composition, the container being closed by a closing member.

The container may be associated with an applicator, for example in the form of a brush comprising an arrangement of bristles maintained by a twisted wire. Such a twisted brush is described in U.S. Pat. No. 4,887,622. It may also be in the form of a comb comprising a plurality of application members, obtained by moulding. Such combs are described, for example, in the document FR 2 796 529. The applicator may be solidly attached to the container, as described, for example, in the document FR 2 761 959. For instance, the applicator can be solidly attached to a stem, which is itself solidly attached to the closing member.

The closing member may be coupled to the container by screwing. According to at least one embodiment, the coupling between the closing member and the container can take place other than by screwing, for example via a bayonet mechanism, by click-fastening or by tightening. According to the present disclosure, the term “click-fastening” is understood to mean any system involving the passing of a rim or bead of material by elastic deformation of a portion, such as of the closing member, followed by return to the elastically unstressed position of the portion after the rim or bead has been passed.

The container may be at least partly made of thermoplastic material. Non-limiting examples of thermoplastic materials that may be mentioned include polypropylene and polyethylene.

Alternatively, the container may be made of a non-thermoplastic material, such as glass or metal (or alloy).

The container may be equipped with a drainer located in the region of the aperture of the container. Such a drainer makes it possible to wipe the applicator and, optionally, the stem to which it may be solidly attached. Such a drainer is described, for example, in document FR 2 792 618.

The content of the documents mentioned previously are hereby incorporated by reference into the present patent application.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the invention are approximations, the numerical values set forth in the specific examples are reported as precisely as possible. Any numerical value, however, inherently contains certain errors necessarily resulting from the standard deviation found in its respective testing measurement.

The examples that follow are intended to illustrate the invention without, however, being limiting in nature. Unless otherwise indicated, the amounts are given in grams.

### EXAMPLES

### Examples 1 to 4

Mascaras 2 to 4 according to the present disclosure comprised at least one volatile oil, and mascara 1 according to the prior art (not comprising any volatile oil) were prepared.

| | 1 | 2 | 3 | 4 |
|---|---|---|---|---|
| | outside | according | according | according |
| | the | to the | to the | to the |
| | disclosure | disclosure | disclosure | disclosure |
| Carnauba wax | 7.30 | 7.30 | 7.30 | 7.30 |
| Rice bran wax | 7.45 | 7.45 | 7.45 | 7.45 |
| Candelilla wax | 2.50 | 2.50 | 2.50 | 2.50 |
| Beeswax | 6.30 | 6.30 | 6.30 | 6.30 |
| Gum Arabic | 1.50 | 1.50 | 1.50 | 1.50 |
| Hydroxyethylcellulose | 0.22 | 0.22 | 0.22 | 0.22 |
| Hydroxyethylcellulose | 0.10 | 0.10 | 0.10 | 0.10 |
| quaternized with | | | | |
| 2,3-epoxy- | | | | |
| propyltrimethyl- | | | | |
| ammonium chloride | | | | |
| Mixture of | 0.12 | 0.12 | 0.12 | 0.12 |
| polydimethylsiloxane | | | | |
| and hydrated silica | | | | |
| Non-stabilized sodium | 1.00 | 1.00 | 1.00 | 1.00 |
| polymethacrylate at | | | | |
| 25% in water (Darvan | | | | |
| 7 from Vanderbilt) | | | | |
| Isododecane | — | 10.00 | — | — |
| Heptamethylethyl- | — | — | 10.00 | — |
| trisiloxane | | | | |
| Heptamethylbutyl- | — | — | — | 10.00 |
| trisiloxane | | | | |
| Pigments | 8.00 | 8.00 | 8.00 | 8.00 |
| Stearic acid | 5.45 | 5.45 | 5.45 | 5.45 |
| Triethanolamine | 2.4 | 2.4 | 2.4 | 2.4 |
| Preserving agents | qs | qs | qs | qs |
| Water | qs 100 | qs 100 | qs 100 | qs 100 |

For each composition, the water resistance and the sebum resistance were measured according to the measuring methods described previously in the description.

The in vitro charging was measured by gravimetry on samples of curly Caucasian hair (30 hairs 1 cm long spread over a distance of 1 cm).

The sample was made up by performing three sets of 10 sweeps of mascara at two-minute intervals, with uptake of product between each series of 10.

The sample was dried for 20 minutes at room temperature and then weighed.

This measurement was performed on six samples.

The charging is the amount (in mg) of material deposited on the sample, i.e., charging =mass of made-up sample−mass of naked sample.

The mean charging is the mean of the measurements taken on the six samples.

The following results were obtained:

| | 1 | 2 | 3 | 4 |
|---|---|---|---|---|
| in vitro charging (mg) | 9.3 | 10.9 | 11.7 | 11.4 |
| Water resistance (ΔL) | −5.1 | −2.31 | −0.24 | −1.5 |
| Sebum resistance (ΔL) | −2.7 | −2.35 | −1.9 | −1.9 |

It was found that the mascaras of Examples 2 to 4 according to the present disclosure have a water resistance and a sebum resistance greater than the mascara not comprising any volatile oil (Example 1), and also higher in vitro charging, and thus a higher charging effect.

What is claimed is:

1. A keratin fiber coating composition comprising a continuous aqueous phase and at least one volatile oil,
wherein when the composition forms a film on the keratin fiber, it has a water resistance such that ΔL is greater than or equal to −4.5,
the at least one volatile oil being chosen from isododecane, 3-butyl-1,1,1,3,5,5,5-heptamethyltrisiloxane, 3-ethyl-1,1,1,3,5,5,5-heptamethyltrisiloxane and octamethyltrisiloxane.

2. A keratin fiber coating composition comprising a continuous aqueous phase and at least one volatile oil,
wherein when the composition forms a film on the keratin fiber, it has a sebum resistance such that ΔL is greater than or equal to −2.5,
the at least one volatile oil being chosen from isododecane, 3-butyl-1,1,1,3,5,5,5-heptamethyltrisiloxane, 3-ethyl-1,1,1,3,5,5,5-heptamethyltrisiloxane and octamethyltrisiloxane.

3. A keratin fiber coating composition comprising a continuous aqueous phase and at least one volatile oil,
wherein the composition has a viscosity, measured at 25° C., of less than or equal to 30 Pa·s, and
when the composition forms a film on the keratin fiber, it has a water resistance such that ΔL is greater than or equal to −4.5.

4. A keratin fiber coating composition comprising a continuous aqueous phase and at least one volatile oil,
wherein the composition has a viscosity of less than or equal to 30 Pa·s, and
when the composition forms a film on the keratin fiber, it has a sebum resistance such that ΔL is greater than or equal to −2.5.

5. A keratin fiber coating composition comprising a continuous aqueous phase, at least one volatile oil, and at least one liposoluble or lipodispersible film-forming polymer that is present in a polymer solids amount of less than or equal to 5% by weight, relative to the total weight of the composition,
wherein when the composition forms a film on the keratin fiber, it has a water resistance such that ΔL is greater than or equal to −4.5.

6. A keratin fiber coating composition comprising a continuous aqueous phase, at least one volatile oil, and at least one liposoluble or lipodispersible film-forming polymer that is present in a polymer solids amount of less than or equal to 5% by weight, relative to the total weight of the composition,
wherein when the composition forms a film on the keratin fiber, it has a sebum resistance such that ΔL is greater than or equal to −2.5.

7. The keratin fiber coating composition according to claim 3, wherein the at least one volatile oil is chosen from hydrocarbon-based oils, silicone oils and fluoro oils.

8. The keratin fiber coating composition according to claim 3, wherein the at least one volatile oil is chosen from hydrocarbon-based oils containing from 8 to 16 carbon atoms.

9. The keratin fiber coating composition according to claim 3, wherein the at least one volatile oil is chosen from C₈-C₁₆ isoalkanes of petroleum origin.

10. The keratin fiber coating composition according to claim 3, wherein the at least one volatile oil is chosen from volatile linear alkyltrisiloxane oils of formula (I): 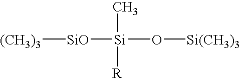
in which R is chosen from an alkyl group containing from 2 to 4 carbon atoms, at least one of the hydrogen atoms of which may be replaced with a fluorine or chlorine atom.

11. The keratin fiber coating composition according to claim 3, wherein the at least one volatile oil is chosen from:
3-butyl-1,1,1,3,5,5,5-heptamethyltrisiloxane,
3-propyl-1,1,1,3,5,5,5-heptamethyltrisiloxane, and
3-ethyl-1,1,1,3,5,5,5-heptamethyltrisiloxane.

12. The composition according to claim 1, wherein the at least one volatile oil is present in an amount ranging from 5% to 40% by weight, relative to the total weight of the composition.

13. The keratin fiber coating composition according to claim 12, wherein the at least one volatile oil is present in an amount ranging from 8% to 15% by weight, relative to the total weight of the composition.

14. The keratin fiber coating composition according claim 1, wherein the at least one volatile oil is present in an amount of at least 5% by weight relative to the total weight of the composition.

15. The keratin fiber coating composition according claim 14, wherein the at least one volatile oil is present in an amount of at least 10% by weight relative to the total weight of the composition.

16. The keratin fiber coating composition according to claim 1, wherein the continuous aqueous phase comprises water and/or at least one water-soluble solvent.

17. The keratin fiber coating composition according to claim 1, wherein the continuous aqueous phase is present in an amount ranging from 5% to 95% by weight, relative to the total weight of the composition.

18. The keratin fiber coating composition according to claim 17, wherein the continuous aqueous phase is present in an amount ranging from 15% to 60% by weight, relative to the total weight of the composition.

19. The keratin fiber coating composition according to claim 1, wherein the continuous aqueous phase is present in an amount of greater than or equal to 20% by weight, relative to the total weight of the composition.

20. The keratin fiber coating composition according to claim 19, wherein the continuous aqueous phase is present in an amount of greater than or equal to 40% by weight, relative to the total weight of the composition.

21. The keratin fiber coating composition according to claim 1, further comprising at least one emulsifying system.

22. The keratin fiber coating composition according to claim 1, further comprising at least one oily-phase structuring agent or organic solvent chosen from waxes, semi-crystalline polymers and lipophilic gelling agents.

23. The composition according to claim 22, wherein the at least one structuring agent is present in an amount ranging from 5% to 80% by weight, relative to the total weight of the composition.

24. The keratin fiber coating composition according to claim 23, wherein the at least one structuring agent is present in an amount ranging from 10% to 55% by weight, relative to the total weight of the composition.

25. The keratin fiber coating composition according to claim 1, further comprising at least one film-forming polymer.

26. The keratin fiber coating composition according to claim 3, further comprising at least one film-forming polymer.

27. The keratin fiber coating composition according to claim 25, wherein the at least one film-forming polymer is present in a solids amount ranging from 0.1% to 30% by weight, relative to the total weight of the composition.

28. The keratin fiber coating composition according to claim 26, wherein the at least one film-forming polymer is present in a solids amount ranging from 1% to 15% by weight, relative to the total weight of the composition.

29. The keratin fiber coating composition according to claim 1, further comprising at least one dyestuff.

30. The keratin fiber coating composition according to claim 29, wherein the at least one dyestuff is present in an amount ranging from 0.01% to 30% by weight, relative to the total weight of the composition.

31. A process for making up keratin fibers, comprising applying to the keratin fibers a composition comprising a continuous aqueous phase and at least one volatile oil,
wherein when the composition forms a film on the keratin fiber, it has a water resistance such that ΔL is greater than or equal to 4.5, the at least one volatile oil being chosen from isododecane, 3-butyl-1,1,1,3,5,5,5-heptamethyltrisiloxane, 3-ethyl-1,1,1,3,5,5,5-heptamethyltrisiloxane and octamethyltrisiloxane.

32. A process for making up keratin fibers, comprising applying to the keratin fibers a composition comprising a continuous aqueous phase and at least one volatile oil,
wherein the composition has a viscosity, measured at 25° C., of less than or equal to 30 Pa·s, and
when the composition forms a film on the keratin fiber, it has a water resistance such that ΔL is greater than or equal to −4.5.

33. A method of forming a keratin fiber makeup composition with a specific water and/or sebum resistance,
comprising adding at least one volatile oil chosen from isododecane, 3-butyl-1,1,1,3,5,5,5-heptamethyltrisiloxane, 3-ethyl-1,1,1,3,5,5,5-heptamethyltrisiloxane and octamethyltrisiloxane, in a keratin fiber coating composition comprising a continuous aqueous phase,
wherein when the composition forms a film on the keratin fibers, it has a water resistance such that ΔL is greater than or equal to −4.5 and/or a sebum resistance such that ΔL is greater than or equal to −2.5.

34. A method of forming a keratin fiber makeup composition with a specific water and/or sebum resistance,
comprising adding at least one volatile oil and at least one liposoluble or lipodispersible film-forming polymer in a polymer solids amount of less than or equal to 5% by weight, relative to the total weight of the composition, to a keratin fiber coating composition comprising a continuous aqueous phase,
wherein when the composition forms a film on the keratin fibers, it has a water resistance such that ΔL is greater than or equal to −4.5 and/or a sebum resistance such that ΔL is greater than or equal to −2.5.
